(19) United States
(12) Patent Application Publication
(10) Pub. No.: US 2026/0315227 A1
(43) Pub. Date: Oct. 15, 2026

(54) BIDIRECTIONAL BLOOD FLOW ATRIAL SEPTAL SHUNT

(71) Applicant: BANDULA WIJAY, Friendswood, TX (US)

(72) Inventor: BANDULA WIJAY, Friendswood, TX (US)

(21) Appl. No.: 19/175,195

(22) Filed: Apr. 10, 2025

(51) Int. Cl. A61F 2/24

(52) U.S. Cl. CPC A61F 2/2476; A61F 2/2418; A61F 2/243; A61F 2210/0009; A61F 2210/0014; A61F 2210/0057; A61F 2210/0076

(57) ABSTRACT

A bidirectional valve device is implanted using minimally invasive techniques in the atrial septum whereby it reduces the atrial blood pressure. The valve adapts to differential pressure gradients through the incorporation of a plurality of valve leaflets, each designed to open at distinct pressure thresholds and having the ability to open in both directions, allowing bidirectional flow, depending on the direction of the pressure gradient. The valve leaflets are sensitive to varying levels of pressure, allowing the valve to open only when a specific pressure gradient is exceeded between the inlet and outlet. Each leaflet or flap operates independently, enabling tuned management of atrial pressures, adapting dynamically to changing hemodynamic conditions within the heart.

### FIELD

The present application generally relates to devices employed in the treatment of heart disease. In particular, the disclosure is directed at a bidirectional heart valve that regulates blood flow in a manner to achieve optimization of the regulation the pressure of blood flow while offering a controlled, bidirectional flow to accommodate physiological needs.

### BACKGROUND

Congestive Heart Failure (CHF) is a leading cause of morbidity and mortality worldwide. CHF is a chronic, progressive condition in which the heart's ability to pump blood is insufficient to meet the body's needs. It often leads to a buildup of fluid (congestion) in the lungs, liver, abdomen, and legs, resulting in symptoms such as shortness of breath, fatigue, and swelling (edema). CHF can affect the left side, right side, or both sides of the heart.

Certain underlying cardiovascular conditions impair the normal function of the heart leading to CHF. Some of these conditions include Coronary Artery Disease (CAD); Hypertension (High Blood Pressure); Valvular Heart Disease; Cardiomyopathy; Myocardial Infarction (Heart Attack); and Arrhythmias. Each of these conditions increase the risk of developing CHF due to their impact on blood vessels and heart function. Many conditions can be treated early but often go undetected or untreated resulting in CHF. This necessitates that not only must the underlying condition be treated but also the downstream adverse effects on the heart.

CHF, or left-sided heart failure is classified as diastolic or systolic heart failures. Systolic heart failure is heart failure with reduced ejection faction (HFrEF). This occurs when the left ventricle loses its ability to contract and pump oxygenated blood from the heart. Diastolic heart failure is heart failure with preserved ejection fraction (HFpEF). The heart experiences elevated left atrial (LA) pressure due to impaired ventricular filling, leading to pulmonary congestion and associated symptoms like dyspnea, fatigue, and exercise intolerance. Traditional heart failure therapies focus on medical management, but for advanced cases, they often fail to adequately relieve symptoms or prevent hospitalizations.

In response, interatrial shunt devices have been developed as a novel mechanical approach to reduce LA pressure. This is accomplished by creating controlled communication between the left atrium (LA) and right atrium (RA) whereby blood flows from the high-pressure LA to the lower-pressure RA. The RA is more capable of handling this increased blood volume and usually tolerates it well.

An example of a shunt is shown in U.S. Pat. No. 10,179,026. This patent describes a non-valvular shunt that creates a permanent opening in the atrial septum, allowing continuous flow between the LA and RA. The lack of a valve mechanism, however, means that flow is not controlled, and complications such as oxygen desaturation and excessive right-to-left shunting can occur.

In order to overcome these limitations valvular shunts have been developed. Two examples are described in U.S. Pat. No. 9,326,928 and U.S. Patent Publication 2005/0165344. This patent and publication describe a unidirectional valvular shunt that allows blood to flow from the LA to the RA, reducing pressure in the left heart. The valves prevent backflow from the RA, which may prevent right atrial overload but lacks the flexibility to allow RA-to-LA flow when needed. One device has a tri-leaflet valve to allow the blood flow from the LA to RA but does not exhibit any control of flow based on the pressure differential, or ability to provide flow from RA to LA when the pressure in the RA is higher due to pulmonary hypertension.

While existing atrial shunt devices as described above provide valuable options for managing elevated left atrial pressure, they are often complex and depend heavily on precise functioning of the elements. In addition, their utility is limited by their inability to allow for or manage bidirectional flow. In certain heart failure patients, particularly those with right-sided heart failure or pulmonary hypertension, pressure in the right atrium may periodically exceed that in the left atrium. This can be alleviated by providing for bidirectional flow. Under these circumstances, an inability to reverse flow can lead to elevated right-sided pressures, contributing to right heart congestion and worsening symptoms.

Thus, a bidirectional shunt device having valving to allow for bidirectional flow would address these clinical gaps by allowing controlled flow from the LA to the RA when left atrial pressure is elevated and enabling flow from the RA to the LA when right atrial pressure exceeds left atrial pressure. This would offer flexibility in the management of complex heart failure cases, providing relief from pressure buildup on both sides of the heart. Additionally, incorporating a flap or mechanism is a simpler design than a tri-leaflet or bi-leaflet valves and is less complicated to manufacture while it can dynamically control the direction and magnitude of flow based on pressure gradients between the atria and would significantly improve patient outcomes. In addition, a valve with a flap system provides for leakage or bypass flow between the two atria to balance the pressure, when the pressure difference is minimum.

### SUMMARY OF THE DISCLOSURE

An apparatus for the regulation of blood flow and pressure is disclosed. The apparatus may be placed in the heart to dynamically regulate blood flow between the left and right atria. The apparatus includes a bidirectional flap valve mechanism or a bidirectional leaflet construction that allows controlled flow of blood between the LA and RA.

The apparatus generally comprises a support or valve frame and a bidirectional valve. The frame is constructed to span an opening in the atrium septum such that it anchors to said septum on both the right and left atrium sides of the opening. The frame includes an opening at its distal end to which the bidirectional valve is mounted or attached or formed integrally with the frame. The valve comprises a flap or leaflet that opens to allow for bidirectional flow, the direction of which depends on the pressure gradient. If the pressure is higher on one side, it will open to allow blood to flow from that side and vice versa if the pressure gradient should change. For example, when right atrial pressure is elevated the flap valve construction, and the bidirectional leaflet valve construction permits flow from the RA to the LA. This manages atrial pressure imbalances, particularly in patients with heart failure and pulmonary hypertension.

The construction of the apparatus may contain two flap members depending on the application. For example, a two-member valve can be provided with one member that opens at a certain pressure gradient and the second member that would open at a different pressure gradient, thereby being able to calibrate the working function of the proposed valve. This configuration may have varying stiffness properties to allow for a rigid to semi-rigid design and includes a living hinge that allows deflection of the valve apparatus. Varying the rigidity and/or including a living hinge is more robust and resistant to mechanical fatigue. This more durable design allows for prolonged use without the degradation that often affects flexible leaflets in leaflet valves.

The members of the bidirectional valve of the present disclosure comprise either flaps or leaflets. For example, the valve may feature at least one flap, where the flap is positioned to provide a separation of fluids in the right and the left atrium. Where two or more flaps are employed, they will open gradually based on the pressure differential between the atria and facilitate flow of blood from the high-pressure area to the low-pressure area at pre-determined stages. Each flap would yield to open at a preset pressure. For example, the first flap will open in the pressure range between 15-20 mmHg, and the second flap will open in the range 20-30 mmHg. Thereby when the pressure is 30 mmHg, for example, both flaps will open and only one will open when the pressure is less than 20 mmHg and greater than 15 mmHg. In yet another feature of the apparatus of the present disclosure, each flap or leaflet can open in a direction either toward the left atrium or the right atrium of the heart. This obviates the need to dedicate a group of flaps for one direction and another group for the opposite direction.

The proposed valve frame and the valve itself can be made from laser cut tube materials of shape memory alloys such as or can be made from shape memory Nitinol wire form. In either case the valve frame is delivered to the opening in the septum by minimally invasive techniques. For example, the valve frame is compressed into a sheath and delivered using a catheter. The wire frame structure, made from laser cut shape or the wire form made using round or flat wire, is heat set to the final desired shape and once removed from a sheath will take the predetermined shape causing it to anchor in the aperture created in the atrial septum.

The valve frame's aperture or opening and the flap valve are covered with a fabric that blocks the flow of blood. This fabric can preferably be attached to the wire frame by using adhesives or by suturing using a thread to attach it uniformly to the wire frame so that the valve diaphragm and the flap structure covering are in place tightly for a leak free flap without any folds or slack in them. Alternatively, the material to be used in closing the open windows of the wire frame and the flap can also be molded into place using biocompatible materials such as silicone, PTFE and various grades of polyurethane. Similarly, the material of the valve diaphragm in the flap of the valve can be made from biocompatible materials such as silicone, Teflon, Teflon fabric (e PTFE), Polyethylene, or organic materials such as the properly processed porcine pericardial tissue.

The flap valve structure is so designed to provide a “living hinge” for the flap to swing in one or both directions as described above. The hinge is designed so that a special region of the wire frame structure will bend at a specific pivot point to provide an axis for the deflection so that it can flex in either direction around that pivot. One way to accomplish this characteristic is by thinning the region of the wire frame structure to form the hinge that has a relatively higher degree of flexibility compared to the other regions of the wire frame structure. Therefore, the flap can flex and deform about that axis to provide blood flow to balance the pressure between the chambers. The degree of flexibility shall be predetermined so that the flap will flex at the desired pressure differential.

The valve frame structure may be formed from laser cutting a tube or bending a Nitinol wire to a predetermined shape and heat setting the said structure. As stated, the frame is positioned in the atrial septum, where a hole has been produced by the various standard methods now in practice in interventional cardiology practice. The valve frame has struts that will expand radially around the hole in the atrial septum thereby anchoring the wire frame to the atrial septum. These struts have the shape and stiffness sufficient to reliably anchor the wire frame onto the septal wall.

A valve chamber, where the valve will be housed, is located centrally to the frame. This valve chamber can be an integral part of the previously described frame or can be made separately and attached to the frame by mechanical means or using precision welding techniques. The valve chamber is larger than the aperture or hole. The struts of the frame extend outwards from the aperture first and turns inwards at an angle such that the leaflets or flaps does not rub against the walls of the chamber as they actuate.

The general shape of the bidirectional valve may be circular in nature. However, if one-flap or two-flap valves are designed, it is advantageous to provide a more elliptical or rectangular shape so that it provides a relatively larger area of valve-surface away from the hinge. This larger area therefore will help the deflection with small pressure gradients relatively easier than the triangular-shape flap shape, where the area away from the hinge is relatively small. The bidirectional leaflet valve can be constructed from similar materials mentioned above, but one leaflet is oriented in one direction while the other leaflet is oriented in the other direction.

The foregoing has outlined rather broadly the more pertinent and important features of the device so that the detailed description that follows may be better understood. Additional features will be described hereinafter which form the subject of the claims of the invention. It should be appreciated by those skilled in the art that the conception and the disclosed specific methods and structures may be readily utilized as a basis for modifying or designing other structures for carrying out the same purposes of the device. It should be realized by those skilled in the art that such equivalent structures do not depart from the spirit and scope of the appended claims.

### BRIEF DESCRIPTION OF DRAWINGS

The above and other objects, features and advantages of the present disclosure will be more apparent from the following detailed description taken in conjunction with the accompanying drawings, in which:

FIG. 1 is a sectional view of the heart having the bidirectional valve of the present application.

FIG. 2 is a rotated, sectional view of apparatus (10) of FIG. 1 showing the bi-directional valve of the present application.

FIG. 2A is a view is a view of the valve in FIG. 2 as seen from the right atrium FIG. 3 is a rotated, sectional view of an alternate embodiment of the bi-directional valve of the present application.

FIG. 4 is a view of the embodiment shown in FIG. 3 from the perspective of the right atrium.

FIG. 5 is a top view of the shunt structure that is opened to a flat configuration, demonstrating that it can be made from a wire form, as a flat structure, and rolled to form a cylindrical shape.

FIG. 6 is a top view of an alternate embodiment of the bidirectional valve of the present application.

FIG. 7 is a side view of the valve of FIG. 6 showing the valve in operation from a closed to open position.

FIG. 8 is a rotate, side view of the valve of FIG. 6 showing the leaflets in operation.

FIG. 9 is a rotated side view of yet another alternate embodiment of the present application.

### DETAILED DESCRIPTION

Exemplary embodiments are described herein to provide a detailed description of the present disclosure. Variations of these embodiments will be apparent to those of skill in the art. Moreover, certain terminology is used in the following description for convenience only and is not limiting. For example, the words “right,” “left,” “top,” “bottom,” “upper,” “lower,” “inner” and “outer” designate directions in the drawings to which reference is made. The word “a” is defined to mean “at least one.” The terminology includes the words above specifically mentioned, derivatives thereof, and words of similar importance.

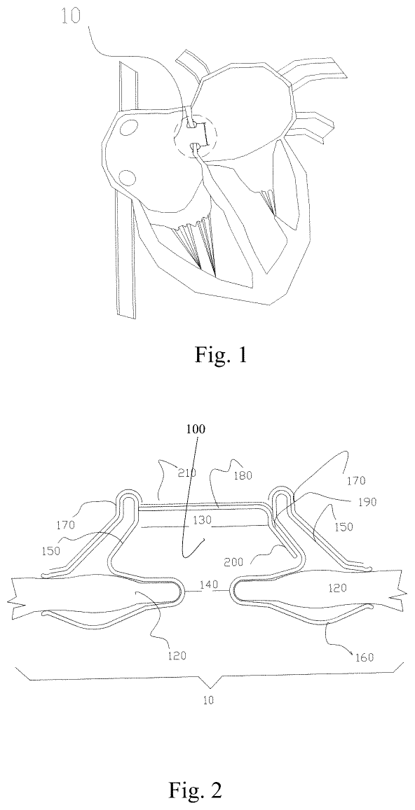

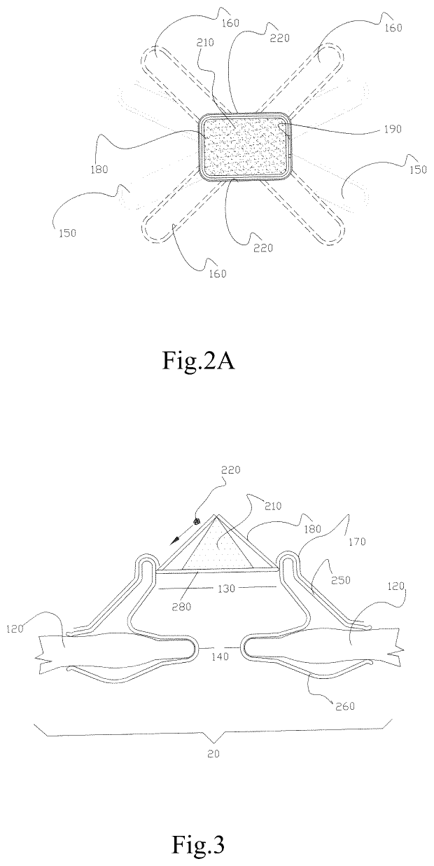

With reference to FIGS. 1 and 2, a bidirectional valve (10) is described in detail below and is implanted in the atrial septum at location at or near fossa ovalis. The bidirectional valve generally comprises an anchoring structure (10) that is anchored to the atrial septum and a valve structure (10). The valve structure (10) can be a bidirectional flap valve or a bidirectional leaflet valve that is attached to the anchoring structure. As seen in FIG. 2, the structure (10) is implanted into the wall of the atrial septum (120), having valve chamber that has an opening (130) which is larger than the area of an aperture or hole (140) in the atrial septum (120). Structure (10) includes annular struts (150) that define the opening (130). The opening (130), is distal to the septal wall, typically in a range of 5-10 mm.

The shunt structure (10) is held in place by the members (150) at its distal end, on the right atrium side of aperture (140) and by members (160) on its proximal end on the left atrium side. These members (150) have shape memory characteristics to provide reliable clamp force to secure the structure (10) to the septal wall (120). These members (150) form an annular structure when crafted by laser cutting the structure (150) from a tube. Alternatively, they can be constructed from a single wire form. The strut members (150) are made by forming it by heat setting a wire form from materials such as Nitinol or is laser cut from a tube and heat set to a predetermined shape using shape memory alloy materials.

Once the shunt structure (10) is placed in situ, tissue will grow into the struts (150) and (160) provide a stable and reliable anchoring of the shunt. The shunt structure (10) is covered by a sheath (170) comprising a biocompatible membrane and is attached either by suturing the membrane to the structure or by molding it on to the structure. The membrane prevents blood leakage. The face of the flap valve (180) is also closed using a biocompatible membrane (210) to block the flow of blood from one side to the other when the valve is in operation. Blood will only flow from the left atrium to the right atrium or vice versa only when the valve opens in the appropriate direction depending on the pressure differential. The membrane may comprise biocompatible polymers like e-PTFE (Expanded Polytetrafluoroethylene), Dacron (Polyethylene Terephthalate), Silicone, porcine or other mammal pericardial tissue, Silicone, and a whole host of materials that have been previously used in artificial heart valves.

The flap valve (180) is placed at the distal end of the structure (10), away from the septal wall, as shown in FIG. 2. The flap valve (180) may take a variety of shapes such as rectangular, elliptical, triangular, or circular shape. The wire structure of the flap valve (180) is attached to the shunt structure (10) either by clamping it on to the latter or by welding it using the extension (190). The extension (190) is either welded or clamped into the structure. Alternatively, flexible wire-member (200) can act as a spring to hold the flap valve (180) in place. Blood will only flow when the valve is open, which only happens when there is a pressure differential.

To place the structure (10) in the atrial septum (120), a hole is made in the fossa ovalis using methods such as puncturing a hole using a catheter and subsequently using a dilation balloon to increase the size of the hole to the desired size. The apparatus (10) is then placed in situ using minimally invasive techniques, typically a catheter system where the device afore mentioned is placed on the outside surface of the distal end of the catheter and a movable sheath is placed over the device compressing it into a transportable form. The catheter is introduced into the vasculature of the patient and advanced to the atrial wall. The sheath is then withdrawn slowly to first expand the distal end of struts (150) in the right atrium. The catheter is slightly moved to properly locate the valve and the sheath is then further pulled back to expand the proximal end of struts (160) into the left atrium thus anchoring the apparatus (10) to the atrial wall.

The structure (10) and valve (180) may be constructed from shape memory alloys, such as Nickel-Titanium alloys, commonly known as Nitinol. In manufacturing the apparatus, the structure (10) can be made from round or flat wire formed into the desired shape and heat setting the structure, so that the desired shape is memorized, so that after the delivery of the device on to the atrial septum, it reverts to the predetermined shape. The apparatus can also be made by laser cutting the structure from a tube of Nitinol or a flat sheet and heat set to the desired shape, at proper heat setting conditions, to impart the physical structure desired for the above purpose. Also, the shunt structure can be made of different components and welded into the desired shape as well before or after the heat setting operation. The membrane described previously can be attached by suturing the membrane to the structure or by molding it on to the structure to make a stable and reliable cover (sheath) over a long period of time.

The structure (10) is anchored in an aperture (140) in the atrial septum (120), as described above. The structure or frame (10) expands such that an opening (130) in the frame will be located at a location where the frame (10) is distal to the aperture (140). The opening (130) is larger compared to the aperture (140) in the septum wall (120). A valve (180) is positioned within opening (130). This size differential between the opening (130) and the aperture (140) is desirable due to the pressure differential needed to open and close the valve is much smaller when the surface area on which the pressure is acting upon is larger.

The struts (150) diverge outwardly from opening (130) and reconverge proximal to the septal wall, preferable on the right atrium side, to form a valve chamber 100. The valve chamber (100) holds the flap valve (180) in place and allows the valve to open in both directions. The divergence of the walls of the chamber (100), formed by the series of struts (150), prevent interference with the motion of the valve (180). In contrast, interference would result if the chamber structure struts were either parallel or converge inwardly toward aperture (140).

FIG. 2A is an illustration of the view of the structure (10) from the right atrium depicting the valve flap (180), which is rectangular in nature. Other shapes such as triangular or elliptical shapes can also be adopted without deviating from the intention of the present invention. The anchoring struts (150) in solid lines are on the right atrium side and the strut members (160) are in the left atrium side. The valve hinges (190) are also shown and are used to attach the flap of the flap valve to the wire frame (220).

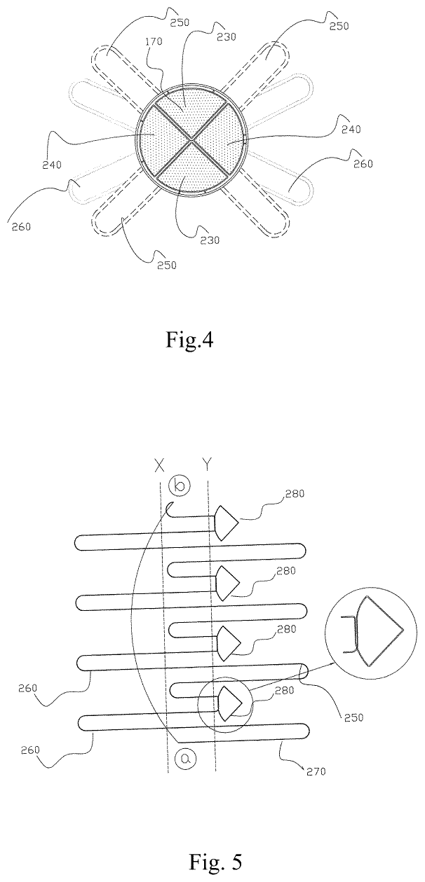

An alternate embodiment of the apparatus (20) is described with reference to FIGS. 3 and 4. The shunt apparatus shown in this illustration has a conical valve (280) having four flaps. In this example the bidirectional atrial shunt valve is implanted into the septum (120) as described above and is held in place by the strut members (250) and (260). The number of strut members (250) can be as few as four members or can be eight members or more depending on the application. The four flaps are sets of two, i.e. (230) and (240). Valve (280) is constructed such that two of the flaps (230) can open outwards and two of the flaps (240) can open inwards depending on the direction of the pressure differential.

The flaps (230) and (240) can be appropriately calibrated to open depending on the desired pressure gradient. This feature will prevent uncontrollable flow by allowing just enough flow based on the pressure differential. The conical valve (280) also has the advantage of preventing any blood clots (220) from right atrium. The conical valve (280) prevents blood clots (220) from entering the left atrium from the right atrium. entering the left atrium. The cone angle can be varied to the desired outcome, is achieved based on space and functionality.

As shown in further detail in FIG. 4, flaps (230) open towards the right atrium and the flaps (240) open towards the left atrium, making this device a bidirectional septal shunt valve. The strut members (250) represented in dash lines are the members in the right atrium that are used in anchoring the shunt to the septum, and the members (260) represented by the solid lines and are in the left atrium and are used in anchoring the shunt to the left atrial side of the septum. The flaps in the flap valve described above are also covered with a sheath (170) to prevent the flow of blood through the flap structure.

FIG. 5 is a representation of the valve (280) when its structure is opened and laid flat. A wire (270) is wrapped as shown in the drawing and is connected from the end of the wire at “a” to the beginning of the wire at “b” to form a circular, elliptical or near rectangular structure. Before the two ends are connected, the wire frame should be bent to 90 degrees at the dotted line X and Y. The triangular portions (281) are bent to 90 degrees away from the plane of the paper and the strut members (250) and (260) are bent to 90 degrees into the plane of the paper. In doing so the four flaps of the structure meet appropriately to form the valve 280, shown on FIG. 3.

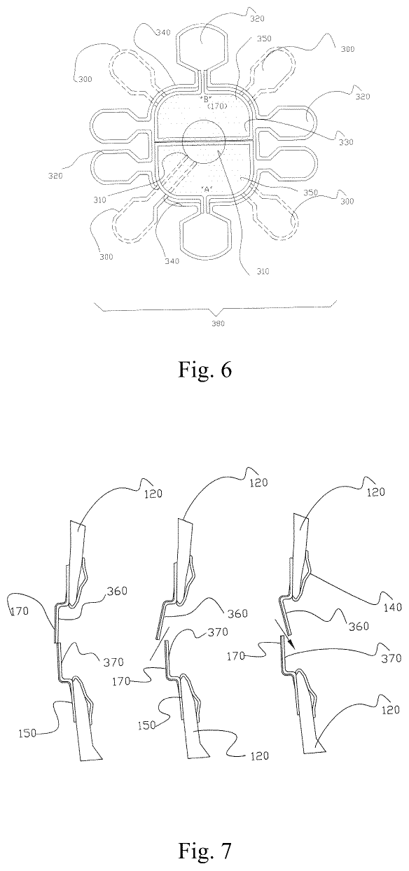

FIG. 6 is a view taken from the perspective of the right atrium and shows an alternate embodiment of a bidirectional valve (380) having two flaps. Strut wire segments (300) pass through the opening in the septal hole (310) from the left atrium to the right atrium and are bent outwards and connect (not shown) into the members (320) that anchors the valve 380. The members (300), shown by dotted lines, are located on the other side of the septum on the right-atrium side. A member (340) forms the valve chamber (330) and defines its shape. The member (340) can be a contiguous part of the strut members (320) and (300) that are functioning as clamps. The flap structure (350) is attached to member (340) at “A” and B”. The flap structure (350) and the valve chamber (330) are encased with biocompatible materials (170) as previously discussed.

The flap structure (350) comprises flaps (360) and (370) and can move either “out of” or “in to” this structure based on the pressure differential. The embodiment in FIG. 6 is shown with two flaps. Therefore, in this embodiment, it is enabled to open at two different pressure gradients, such as from 10-15 mmHg and 15-20 mmHg. While this is an advantage in the proposed design, as the valve opening can be calibrated, a bidirectional single flap embodiment can also be designed based on the proposed invention to accomplish the pressure reduction in either of the atria for the desired clinical outcome.

FIG. 7 illustrates the apparatus of FIG. 6 as it functions in situ. The valve flap (360) is designed to open when the pressure differential is 10-15 mmHg while valve flap (370) is designed to operate when the pressure differential is 15-20 mmHg. Each flap (360) and (370) can also be calibrated to open at other pressure gradients as well. As shown in FIG. 7, the valve can open in either direction-left to right or right to left, based on the pressure gradient.

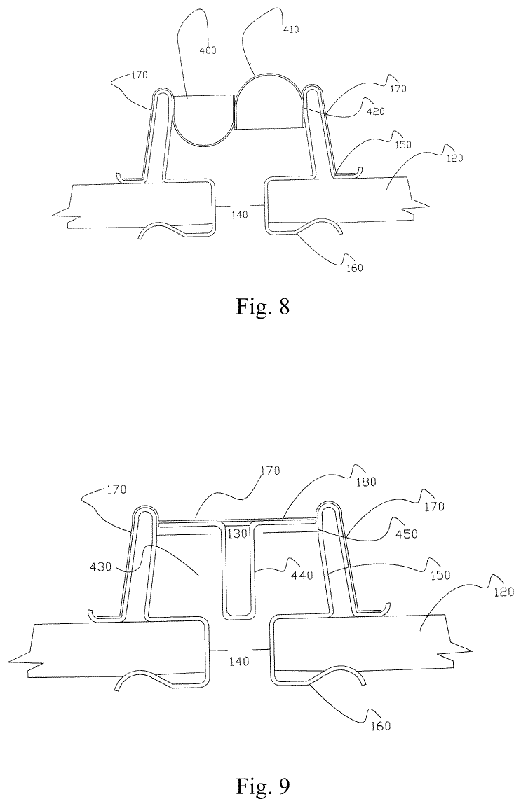

FIG. 8 shows an alternate embodiment wherein the bidirectional leaflet valve is constructed from at least two flexible leaflets (400) and (410). Each is mounted to a shunt structure (420) using appropriate suturing methods to hold the leaflet valve rigidly attached to the structure. The leaflets (400) and (410) are free to open and close based on the pressure differential. The two leaflets are oriented in opposite directions. When the pressure is higher in the left atrium, blood will flow to the right atrium side and vice versa. The construction of the leaflet valve can be made from any of the materials previously mentioned that are biocompatible. The pressure differential at which the leaflet will yield to allow blood flow and can be controlled by the thickness of the material and the elastic modulus of the material used for the construction of the valve. The construction of a bidirectional leaflet valve is less complicated and therefore can be adopted easily to accomplish the need to relieve the adverse pressure conditions in the left or the right atrium.

FIG. 9 shows yet another embodiment of the bidirectional flap valve as described in FIG. 6. The struts are designed to position them converge inwards to prevent hindering or interfering with the valve opening function. The septum (120) has a hole with a relatively small diameter (140) of around 5 mm. There is an advantage in creating a smaller hole in this interventional procedure in the septum, yet large enough that it will not close subsequently due to scar tissue formation. However, the proposed embodiment has an expanded valve chamber (430) having an increased area (130) for placing the valve of to accommodate a larger flap valve. As a smaller flap valve may not permit sufficient blood flow during a single stroke of the heart. In addition, this is a great advantage, in this design, as a smaller flap, due to the smaller surface area of the valve surface will not result in generating enough force to open or close as the force acting on the flap would be small, if the area and the pressure acting on it, is small.

Configuring a larger surface area of the valve by using an expanded valve chamber enables and facilitates providing a larger area and therefore higher force on the hinge 440 as in FIG. 9 of the flap valve, making it easier to open and close the valve. The structure of the above design shows a converging view of the strut members on the right atrium side. This feature enables the flap to move in both directions without getting stuck or rubbing against the struts of the valve chamber, due to the fact the gap between the flap and the wall of the septal shunt is increasing towards the septum. When the valve configuration is rectangular or elliptical in nature, the struts in the long sides of the rectangle or ellipse will converge inwards as shown on FIG. 9. However, it is not necessary to have the strut members on the short side of the rectangle to be convergent as these sides will be the side of the valve where the flap of the valve will be attached, i.e. the hinge location.

As shown in the strut design in FIG. 9, the struts are convergent in nature but provide a small distance (450) where the struts are parallel to each other and are perpendicular to the septal wall. FIG. 9 is a view of the atrial septal shunt with bidirectional valve where the valve is built with one flap (180). The anchoring members (not shown) that support the valve are parallel in nature as the valve hinge is in the short side of the rectangular opening, where the shunt struts can be parallel in nature. The flap valve is attached to the struts with members (440). Only two anchoring members are shown in this view but the anchoring members on other sides are not shown for clarity.

The present disclosure includes that contained in the appended claims as well as that of the foregoing description. Although this invention has been described in its exemplary forms with a certain degree of particularity, it is understood that the present disclosure has been made only by way of example and numerous changes in the details of construction and combination and arrangement of parts may be employed without departing from the spirit and scope of the invention.

What is claimed is:

1. An apparatus comprising:
a valve frame adapted for delivery to an atrial septum for secure positioning within an aperture in an atrial septum, said valve frame having an opening in the frame at a position that is distal to the aperture and being larger than said aperture;
a bidirectional valve having at least one flap or leaflet said bidirectional valve being mounted on said frame in said opening; and
wherein when said frame is positioned within said aperture said valve is configured in such a manner as to regulate blood flow between a left atrium (LA) and a right atrium (RA) of the heart.

2. The apparatus claim 1, wherein the at least one flap that opens in a first and second direction, said first direction toward the right atrium (RA) and said second direction toward the left atrium (LA) said direction of opening being determined by the pressure differential between the LA and RA.

3. The apparatus of claim 2, wherein the valve comprises multiple flaps, each configured to open at different predetermined pressure gradients.

4. The apparatus claim 3, wherein a first flap opens when the pressure differential is between 5-10 mmHg, and a second flap opens when the pressure differential is between 10-15 mmHg.

5. The apparatus of claim 1, wherein the valve frame is constructed from a shape memory alloy, such as Nitinol, and is adapted for delivery by a catheter system.

6. The apparatus of claim 1, wherein the valve frame includes a plurality of struts that expand radially upon deployment such that the frame is anchored in said aperture and the opening housing the valve is located distal to the aperture in the atrial septum.

7. The apparatus of claim 1, wherein the bidirectional valve is designed to fully seal when closed to prevent backflow or leakage.

8. The apparatus of claim 7, wherein the bidirectional valve is designed to allow a bypass flow that limits blood stagnation and reduces the risk of clot formation.

9. The apparatus of claim 1, wherein the bidirectional valve includes a hinge mechanism formed by a flexible region of the valve frame to facilitate controlled bidirectional movement.

10. The apparatus of claim 1, wherein the bidirectional leaflet valve comprises a first leaflet that permits blood flow from the LA to the RA when left atrial pressure exceeds right atrial pressure and a second leaflet that permits blood flow from the RA to the LA when right atrial pressure exceeds left atrial pressure.

11. The apparatus of claim 6, wherein the bidirectional valve further comprises a valve chamber formed from the struts, said valve chamber being positioned on the right atrial side of the septum.

12. The apparatus of claim 6, wherein the valve chamber is wherein the struts diverge from the valve chamber is formed from the struts which diverge away from the opening and converge back toward a centerline of the aperture in the atrial septum at a point that is proximal to said atrial septum.

13. The apparatus of claim 1, wherein the valve structure is covered with a biocompatible material selected from a group of silicone, PTFE, polyethylene, or processed porcine pericardial tissue.

14. The apparatus of claim 1, wherein the shape of the valve chamber is a shape that maximizes the effective surface area for controlled deflection under pressure.

15. The apparatus of claim 1, wherein the bidirectional valve is designed to dynamically adjust to real-time atrial pressure fluctuations, thereby optimizing pressure balance between the atria.

16. The apparatus of claim 1, wherein the valve frame includes a neck region that conforms to the aperture while diverging outward to form a larger valve chamber for enhanced function.

17. A method of implanting a bidirectional valve device in an atrial septum, comprising the steps of:
Creating an aperture in the atrial septum;
Compressing the valve device onto a distal end of a delivery catheter;
Placing the delivery catheter into a blood vessel and actuating the delivery catheter through said vessel to the aperture;
Actuating a proximal end of the delivery catheter so as to cause the valve to release and expand in a radial direction from said catheter within the aperture whereby the valve is anchored therein; and
Positioning the valve chamber to ensure dynamic blood flow regulation between the atria.
